(19) United States
(12) Patent Application Publication
(10) Pub. No.: US 2015/0148573 A1
(43) Pub. Date: May 28, 2015

(54) OLIGOMERISATION OF OLEFINS FOR THE PRODUCTION OF SYNTHETIC FUEL

(71) Applicant: IGTL TECHNOLOGY LTD, Aberdeen (GB)

(72) Inventors: Richard Hyman, Aberdeen (GB); Alfred Ecker, Wien (AT)

(73) Assignee: IGTL TECHNOLOGY LTD, Aberdeen (GB)

(21) Appl. No.: 14/402,526

(22) PCT Filed: May 21, 2013
(86) PCT No.: PCT/GB2013/051334

§ 371 Date: Nov. 20, 2014

(30) Foreign Application Priority Data
May 21, 2012 (DE) 102012208417.7

(51) Int. Cl. C10L 1/04; C07C 2/12

(52) U.S. Cl. CPC C10L 1/04; C07C 2/12; C10L 2200/0469; C10L 2270/04; C07C 2529/06
USPC 585/16; 585/700

(57) ABSTRACT

The invention relates to a synthetic hydrocarbon fuel composition, and a process for making such a fuel composition, in which the fuel composition has the following properties:
a) a boiling point distribution having (i) a 10% recovery of 205° C. or less and (ii) an end point of 300° C. or less;
b) a freezing point of −47° C. or less; and
c) a density at 15° C. of at least 775 kg/m³.

A process for producing the fuel composition comprises the oligomerisation of olefins over an oligomerisation catalyst. In addition the invention relates to a process for producing hydrocarbons comprising contacting one or more olefins with an oligomerisation catalyst in a reaction zone under conditions sufficient to cause oligomerisation of olefins, and removing an outlet stream comprising products of olefin oligomerisation from the reaction zone, wherein the oligomerisation catalyst comprises a crystalline zeolite Brønsted acid catalyst, whose crystals comprise an inorganic oxide framework with an internal pore structure, and an external surface, in which the ratio of the number of Brønsted acid sites on the external surface compared to the internal pore structure is in the range of from 0.1 to 20% and/or the ratio of the surface area of the internal pore structure compared to the external crystal surface area is in the range of from 5 to 1000.

### FIELD OF THE INVENTION

The present invention relates to a hydrocarbon composition suitable for use as a fuel, and which is derived from sources other than crude oil (so-called “synthetic” fuel). The invention in particular relates to a multipurpose hydrocarbon fuel that can be used for a variety of applications, especially as a jet fuel, without the need for blending. The invention also relates to a process for making such a hydrocarbon composition.

### BACKGROUND TO THE INVENTION

Hydrocarbon fuels are typically derived from the refining of crude oil. Hydrocarbon fuels can also be made from alternative sources, for example through Fischer-Tropsch synthesis from syngas (a gaseous mixture comprising carbon monoxide and hydrogen). The syngas can be derived from a variety of sources, for example coal, biomass, natural gas, and carbon-containing domestic and commercial waste. Typical processes for converting such materials to syngas include partial oxidation, steam reforming and autothermal reforming.

Fuels made from such alternative processes, i.e. which are not made from crude oil refining, are often referred to as “synthetic fuels”.

There are a number of different types of hydrocarbon fuel, formulated for different end use purposes. Examples include gasoline, diesel, jet fuel, aviation gasoline and marine fuels, and the related specifications associated with such fuels are intended to ensure a minimum level of performance of the engines or turbines to which they will be fed.

In certain environments, for example in remote locations, or locations that have become isolated as a result of a natural disaster for example, it is logistically complicated to obtain and transport a variety of different types of fuels.

US 2012/101317 describes a method of preparing a joint synthetic fuel formulation for jet, rocket or diesel engines, containing a blend of so-called “high density synthetic” and “low density synthetic” fuels. However, this still requires the production and preparation of two different fuels that require blending.

U.S. Pat. No. 4,720,600 describes an oligomerisation process for converting lower olefins to distillate range hydrocarbons that can be used as jet fuel.

However, there remains a need for a synthetic hydrocarbon fuel and a process for making such a fuel that has the boiling point, density and freezing point characteristics that make it suitable for a variety of applications, including for use as a jet fuel and as a diesel fuel.

### SUMMARY OF THE INVENTION

According to the present invention, there is provided a synthetic hydrocarbon fuel composition which has the following properties:
(a) a boiling point distribution having (i) a 10% recovery of 205° C. or less and (ii) an end point of 300° C. or less;
(b) a freezing point of −47° C. or less; and
(c) a density at 15° C. of at least 775 kg/m³.

The invention also relates to a method for preparing such a hydrocarbon mixture from which the fuel composition can be obtained, in which one or more olefins are contacted with an oligomerisation catalyst in a reaction zone under conditions sufficient to cause oligomerisation of olefins, and removing from the reaction zone an outlet stream comprising products of olefin oligomerisation, wherein a fraction is obtained from the outlet stream which, optionally after hydrotreatment, has the above properties.

The present invention also provides a process for preparing hydrocarbons comprising contacting one or more olefins with an oligomerisation catalyst in a reaction zone under conditions sufficient to cause oligomerisation of olefins, and removing from the reaction zone an outlet stream comprising products of olefin oligomerisation, wherein the catalyst comprises a crystalline zeolite Brønsted acid catalyst, whose crystals comprise an inorganic oxide framework with an internal pore structure, and an external surface, in which the ratio of the number of Brønsted acid sites on the external surface compared to the internal pore structure is in the range of from 0.1 to 20% and/or the ratio of the surface area of the internal pore structure compared to the external crystal surface area is in the range of from 5 to 1000.

An advantage of the present invention is that a single synthetic hydrocarbon fuel composition can be obtained that can be used, without the need for blending, as a jet fuel, and also as a diesel fuel.

Hydrocarbon fuels, whether synthetic or whether derived from crude oil, typically either have insufficient cold flow properties, or have a density (or specific gravity) that is lower than jet fuel specifications require. This means that they are not capable of working efficiently at cold temperatures, as is required for example for aviation fuels or for winter or arctic diesel fuels, or they do not have a density high enough for use in aviation purposes without being blended with higher density compositions. The present hydrocarbon composition achieves both these aims, without the need for blending. It can be used in its own right without blending, or it can alternatively be blended with one or more other sources of fuel.

Because the fuel according to the present invention is multi-purpose, planning and implementing operations in crisis areas or in remote locations is easier, and permits more efficient storage and distribution of fuel.

The term “synthetic fuel” as used herein refers to a fuel whose hydrocarbons are derived or prepared from a process other than the refining of crude oil. In the process of the present invention, the hydrocarbons are the products of olefin oligomerisation, or are derived from the products of olefin oligomerisation.

### DETAILED DESCRIPTION OF THE INVENTION

In the process of the present invention, one or more olefins are oligomerised over an oligimerisation catalyst. This is typically a Brønsted acid catalyst. The products and by-products of the catalytic oligomerisation of olefins are highly influenced by the catalyst used in the process. Thus, not only do acid catalysed oligomerisation reactions take place, where two or more molecules are combined to form a larger molecule, but also isomerisation reactions, hydrogen transfer reactions and cracking reactions. Isomerisation reactions involve the transformation of a molecule into another molecule of the same molecular weight but different configuration (for example a linear to a branched hydrocarbon). Hydrogen transfer reactions involve the removal of hydrogen from a hydrocarbon molecule to another molecule or to the catalyst. Cracking reactions involve the transformation of a molecule into two or more molecules of lower molecular weight.

The catalyst in one aspect of the process of the present invention is a crystalline zeolite catalyst exhibiting Brønsted acidity. Zeolites have a defined crystalline structure. The crystals comprise an external surface, and a porous intracrystalline structure formed within an oxide framework. Typical oxides that can adopt a zeolite structure include silicates, aluminosilicates, aluminium phosphates, aluminosilicophosphates, germanosilicates, gallosilicates and gallophosphates. The acidic zeolites have a net-negative charge on the framework, for example in the case of aluminosilicates, silicoaluminophosphates and gallosilicates. Aluminosilicates are preferred in the present invention.

The pores are made up of channels, whose diameter can be defined by the number of vertex atoms that surround the channel. For example, a so-called “10-membered ring” window comprises 10 vertex atoms. Vertex atoms are atoms other than oxygen present in the zeolite framework. For example, the vertex atoms of aluminosilicates are Al and Si. For silicoaluminophosphates, the vertex atoms are Si, Al and P. In the present invention, the zeolite preferably has channels defined by 12-membered rings or 10-membered rings.

Where channels intersect, for example in 2-dimensional or 3-dimenstional porous networks, they form so-called “cages”, linked via so-called “windows”. The windows typically have a lower diameter than the cages. As with channels, the size of the windows can be defined by the number of atoms forming the window. Typically, windows or channels are considered to be a window or channel they are formed of 8-or more membered rings. For improved diffusion of reactants and products, the pore channels of the zeolite are preferably 2- or 3-dimensional, preferably 3-dimensional.

In preferred zeolites, the maximum window or channel size in all dimensions is a 10-membered ring. Preferably, the window and/or channel diameter is 0.6 nm or less in all dimensions.

Types of zeolite structure that are useful in the present invention include those that adopt the MFI structure of which ZSM-5 is an example, the MEL structure of which ZSM-11 is an example, the MTW structure of which ZSM-12 is an example, the MTT structure of which ZSM-23 is an example, and the FER structure of which ZSM-35 and ZSM-38 are examples. These structures are defined in the International Zeolite Association database of zeolite structures.

In zeolites, Brønsted acid sites exist on the external surface of the crystals and also within the internal pore structure of the zeolite crystals. These different acid site locations impart differences to the reactivity of the acid sites. Reactions on external acid sites are not sterically hindered and therefore they can catalyse oligomerisation reactions, isomerisation reactions, hydrogen transfer and cracking, whereby larger molecules can be formed, for example large polycyclic and aromatic and non-aromatic molecules and coke precursors. Reactions on internal acid sites are constrained by the pore size and channel structure of the zeolite, and therefore these reactions can only produce products with limited size and number of side chains, for example monocyclic aromatic and non-aromatic compounds, and unbranched hydrocarbons or hydrocarbons with small branching groups, for example methyl groups.

In terms of acidity, the zeolite needs to have a balance of sufficient numbers of Brønsted acid sites, while not causing excessive formation of less desired products, for example polyaromatics or highly branched hydrocarbons. This can be achieved by the zeolite having a molar ratio of silicon to aluminium atoms of at least 45, more preferably at least 80 and still more preferably 100 or more. To maintain sufficient numbers of acid sites for reaction to occur, the ratio is also preferably no more than 300. If the Si/AI ratio is too low, formation of less desired polyaromatic and highly branched hydrocarbons is increased. In addition, reaction within the zeolite pores can be inhibited due to the blocking effect of oligomers adsorbed on neighbouring acid sites. If the Si/AI ratio is too high, then reaction rates suffer. High Si/AI ratios can also affect product selectivity, because the strength of Brønsted acid sites in high Si/AI zeolites tends to be higher on average, which can also lead to the formation of less desired products.

The MFI structure has a 3-dimensional pore channel structure, in which the windows in all three dimensions have 10-membered rings. In the zeolite ZSM-5, the aluminosilicate framework has a 3-dimensional structure, with 10-ring windows in all three dimensions, the window diameter being 5.4 to 5.6 Å depending on the dimension. Therefore, ZSM-5 is a suitable catalyst for use as an oligomerisation catalyst in the process of the present invention, and can be tailored to have the above-mentioned ranges of ratios of framework silicon to aluminium atoms.

Another way of achieving the correct distribution of products is to control the number of acid sites within the pore channels compared to the number of acid sites on the external surface of the zeolite crystals. As mentioned above, the products formed as a result of acid-catalysed reactions (including oligomerisation reactions) within the zeolite channels are affected inter alia by the constraints imposed by the zeolite framework, pore, window and cage structure. However, on the external surface of the zeolite crystals, there is no such constraint or control. Often this results in the production of excessive quantities of polyaromatics or highly branched products. Highly branched hydrocarbons, although benefitting low freezing point characteristics of fuels, tend also to reduce cetane number. Polyaromatics tend to have poorer combustion characteristics, and can lead to coking and deactivation of the catalyst.

In the present invention, the hydrocarbon product of the process preferably has branched hydrocarbons, although the number of branches is preferably, low, typically 1 to 2 branches per molecule, and preferably the branches have short chains, preferably having no more than 1-2 carbon atoms per branch, and are more preferably methyl branches. By limiting the extent of branching and the size of the branches, the required cetane numbers and cetane index of resulting fuels can be maintained, while the cold flow properties and freezing point properties of the fuel are acceptable for aviation and/or winter or arctic environments. In addition, branched hydrocarbons tend to increase density compared to unbranched hydrocarbons.

The crystallite size and the Si/AI ratio of the catalyst, as well as the reaction period, temperature and pressure and the recycling ratio of fresh feedstock to the recycled proportion and the composition of the feedstock mixture, can all contribute to the resulting product composition, and therefore the properties of the end synthetic hydrocarbon product.

One way of controlling the ratio of external to internal acidic sites is to use larger zeolite crystals, such that the ratio of external surface area of the crystals compared to the total surface area within the pores is reduced. However, the crystal sizes must also not be so large as to limit the reaction rate too severely due to intra-particle diffusion restrictions of reactants and products in the narrow pore channels. In the oligomerisation catalyst of the present invention, the ratio of internal (intra-pore channel) surface area compared to the surface area of the external surface of the primary crystals is preferably in the range of from 5 to 1000, more preferably from 8 to 300, and more preferably from 10 to 130.

In preferred embodiments, the average primary crystal size of the zeolite (for example ZSM-5 zeolite) is in the range of from 0.1 to 0.9 μm, preferably from 0.2 to 0.8 μm. By primary crystal size is meant the average size of unagglomerated crystals. ZSM-5 crystals having a range of from 0.1 to 0.9 μm typically have an internal pore surface area to external surface area ratio in the range of from 12.3 to 120.

Another way of controlling the ratio of internal to external acid sites is to deactivate selectively the acidic sites on the external surface of the crystals. This can be achieved, for example, by using bulky basic molecules, for example amines or pyridines such as collidine, that are too large to enter the pores. However, such neutralisation techniques may not be stable under oligomerisation conditions, for example due to leaching or dissociation of the basic molecule. In addition, as explained further below, there is an advantage in having at least some acidic sites still present on the external surface of the zeolite crystals. Another means of neutralising external acidic sites that can be more stable to oligomerisation reaction conditions is to use large metal cations, for example rare earth cations (lanthanides). However, the cations are still sufficiently small to migrate into the porous network, reducing the selectivity of external acid site deactivation.

In an alternative embodiment, agglomerates of zeolite crystals can be produced. In one aspect, this can be achieved during synthesis of the zeolite by adding a surface tension modifying agent, such as an acrylamide copolymer or cationic acrylic acid derivative, to the synthesis gel. After any drying, calcination and ion exchange (to form protonated sites) processes, a zeolite having agglomerated primary crystals is formed. Where the zeolite crystals combine or bond together, this reduces the external crystalline surface area available for reaction or for the ion exchange, which results in an improved ratio of intra-pore/intra-crystal acid sites compared to external acid sites that are available for reaction. At the same time, by maintaining small primary crystals, limitation of reaction rates through intra-crystalline diffusion restrictions is avoided. In the agglomerated crystallites, meso- and macropores exist between the crystallites which are substantially larger than the pore windows and channels within the internal framework of the zeolite crystal structure. These larger meso- and macropores do not inhibit diffusion of reactants and products to and from the external surfaces of the primary zeolite crystals, and hence do not reduce reaction rates.

Where zeolite crystal external surface area is referred to, it means the surface area of the zeolite primary crystals that are exposed to reactants on the external surface of any aggolomerates, and with the meso- and macroporous pores and voids between the agglomerated primary crystals.

Typically, where agglomerated zeolite crystals are used, the average size of the agglomerates are in the range of from 80 to 200 μm.

Zeolite crystal size and agglomeration can be controlled by selecting appropriate synthesis conditions. Zeolite crystal size can be controlled by temperature and aging time, such that higher temperatures and aging times tend to lead to larger crystals. Maintaining temperatures less than 175° C. helps to maintain smaller crystal sizes, and increase agglomeration. The rate of agitation or mixing of the synthesis gel can also impact crystal size and agglomeration. Higher rates of stirring and/or increased agitation tend to result in smaller crystallite size, and reduced agglomeration.

The proportion of external surface acidity compared to intra-pore acidity (in terms of the number of Brønsted acid sites) is preferably in a range of from 0.1 to 20%, more preferably 0.3-12.5%, and even more preferably 0.3 to 5%. It can be advantageous to maintain some external acidity, as some products produced by reactions at the external surface, for example isomerisation or (to some extent) aromatic, cracking and branching reactions, can confer certain benefits to the product, for example increasing the extent of branching to improve low temperature properties (isomerisation), controlling hydrocarbon size (cracking) and increasing density (formation of aromatics and cyclic hydrocarbons). The presence of aromatics in the fuel product can help maintain the required density (at 15° C.) of at least 775 kg/m³, and also help maintain desired seal swell and lubricity properties. The proportion of external to intra-pore acid sites can be determined by the method of Zhao et al, as reported in J. Phys. Chem. B, 2002, 106, pages 4462-4469. Note that densities are sometimes expressed in terms of specific gravity, which is a measure of the density of the substance at a specified temperature compared to the density of water at 4° C. A density of 775 kg/m³ at 15° C. is equivalent to a specific gravity of 0.775.

In the oligomerisation process, one or more olefins are contacted with the catalyst in a reaction zone (typically a reactor). Typical process conditions in the reaction zone include a reaction zone or reaction zone inlet temperature of 180 to 350° C., for example 180 to 280° C., such as a temperature in the range of from 220 to 260° C. Preferably, the pressure in the reaction zone is at least 30 bar (3.0 MPa), and preferably greater than 50 bar (5.0 MPa). More preferably, the pressure is in the range of from 30 to 60 bar (3.0 to 6.0 MPa). An outlet stream is removed from the reaction zone. One or more recycle streams can be produced from the outlet stream. For example, a fraction boiling at 180° C. or less, for example 170° C. or less, or 160° C. or less, can be recycled back to the reaction zone to ensure hydrocarbons of appropriate length are produced, and to maximise efficient use of the feedstocks. Deactivation of the catalyst over time can be compensated by increasing the reaction temperature and/or pressure.

Alpha-olefins are the preferred feedstock for oligomerisation in order to avoid excessive branchings in the iso-paraffins. Iso-olefins can be tolerated, although their presence is preferably controlled to avoid the formation of too many and too highly branched hydrocarbons in the resulting product. In one embodiment, the content of iso-olefins in the feedstock is maintained at a value of 10 mol % or less. Additionally, because propene tends to introduce methyl branches into oligomerised hydrocarbons, it is preferably maintained at a value of 30 mol % or less in the feedstock to control the extent of branching in the hydrocarbon products. Iso-olefins can be present at higher values. For example, the dehydration of fusel oil results in a high proportion of iso-olefins which can still produce a synthetic hydrocarbon fuel product according to the present invention.

Propene is a reactive olefin, and hence its presence can be advantageous. However, it can cause the production of iso-olefins with a high degree of methyl branching. To maintain a low number of branchings, preferably no more than 30% propene should be contained in the oligomerisation feedstock. Iso-olefins also display high reactivity, and can produce products with a high degree of substitution. In one embodiment, their concentration in the feedstock mixture is no more than 10% to maintain high cetane number so that the fuel can be used as a diesel fuel.

Iso-olefins are formed from alpha-olefins during the oligomerisation reaction. To increase the degree of conversion and maximise the yield of middle distillate, a light proportion of oligomer formed (up to 180° C., preferably 160° C.) can be recycled. The weight ratio of recyclate to fresh feedstock is between 0.5 and 2.5 and preferably between 1 and 2. To limit the proportion of iso-olefins with too high a degree of substitution (proportion of side chains) formed by recycling, the composition of the feedstock mixture is preferably carefully balanced with the proportion of recyclate. For example, the higher the proportion of olefins with a higher C number (number of carbon atoms) in the feedstock mixture, the more readily iso-olefins with a lower degree of branching are formed, and a lower recyclate/feedstock ratio can therefore be used.

As the carbon number increases, the oligomerisation activity decreases. Where there are high proportions of alpha-olefins having >4 C atoms, the reaction period is preferably extended, i.e. the mass flow per mass of catalyst per hour (WHSV=weight hourly space velocity) is preferably reduced. Preferably, WHSV is in a range of from 0.4 to 1 h⁻¹. In one embodiment, the WHSV is about 0.5 h⁻¹.

The proportion of C6+ hydrocarbons in the feedstock not only influences the reactivity but, in the event of incomplete conversion, requires greater hydrogenation outlay to saturate the unreacted olefins and greater distillate outlay. Therefore, in one embodiment, C6+ hydrocarbons constitute no more than 20% of the feedstock.

In order to achieve a suitable chain length with a reduced degree of branching and low polyaromatic formation during the oligomerisation of alpha-olefins, the following measures are also helpful: increasing the pressure to >50 bar, reducing the temperature to <280° C., increasing the reaction period (decreasing the weight hourly space velocity), increasing the proportion of alpha-olefins with 4-6 C atoms in the feedstock, increasing the crystallite size of the zeolite, adjusting the feedstock composition and the recycled proportion. Increasing the size of particles of agglomerated zeolite crystals can also help achieve this.

A variety of olefin sources can be used in the process of the present invention. In one embodiment, for example, the olefins can be produced by a Fischer-Tropsch process. The source of the syngas used in the Fischer-Tropsch process can be a so-called “stranded” hydrocarbon supply, for example remote natural gas or shale gas sources. Alternatively, it can be derived from a mineral coal or crude oil supply. In further embodiments, the syngas can be produced from gasification of biomass or municipal waste. Another source of olefins is from the dehydration of alcohols, for example alcohols derived from fermentation such as bioethanol or biobutanol, or fusel oil.

The olefin feedstock typically predominantly comprises one or more olefins having from 2 to 10 carbon atoms. Preferably, the majority of olefins in the feedstock comprise 8 carbon atoms or less. In embodiments, the olefin feedstock comprises less than 10 mol % olefins with 10 or more carbon atoms, more preferably less than 5 mol %. Preferably, the olefin feedstock comprises less than 5 mol % branched hydrocarbons having 12 or more carbon atoms. The olefinic feedstock preferably comprises less than 20 mol % paraffins. Preferably, the polycyclic aromatics content of the olefinic feedstock is less than 10 mol %, and more preferably less than 3 mol %. The content of olefins having 6 or more carbon atoms in the olefinic feedstock is preferably no more than 20 mol %. In this context, feedstock does not include olefins recycled from the outlet stream to the reaction zone.

The process of the present invention typically produces hydrocarbons having from 5 to 22 carbon atoms. Typically, the synthetic hydrocarbon fuel composition can be obtained by fractionating or distilling the hydrocarbons obtained from the oligomerisation process of the present invention. In one embodiment, the obtained hydrocarbons are hydrogenated or hydrotreated before the synthetic hydrocarbon fuel is produced. Hydrogenation treatment saturates unreacted olefins, which can deteriorate the quality of the fuel over time. In addition, aromatics can be hydrogenated to cyclic alkanes. Cyclic alkanes can also help maintain the required density of the fuel. Hydrotreatment can be carried out at temperature in the range of from 200 to 300° C., for example in the range of from 230 to 270° C. The pressure can be in the range of from 40-80 bar (4.0 to 8.0 MPa), for example in the range of from 55 to 65 bar (5.5 to 6.5 MPa). A nickel or nickel-containing catalyst can be used. More severe reaction conditions tend to result in greater conversion of olefins and aromatics to alkanes and cyclic alkanes respectively.

The content in aromatics of the final fuel can be tailored by adjusting the severity of the hydrotreating step.

The requirements of the feedstock composition, process parameters and catalyst defined according to the invention make it possible to produce a hydrocarbon product which can be used to make a fuel that meets the requirements for aviation jet fuel, while at the same time being suitable for use in diesel engines. The hydrocarbon composition resulting from the process of the present invention can be used to prepare (for example by distillation) a fuel having the combined properties of a freezing point of less than −47° C., and density of at least 775 kg/m³ that is required for a jet fuel, and which has a upper (end) boiling point of 300° C., without the need for it to be blended with conventional fuel sources. Other non-blended, synthetic hydrocarbon compositions do not have a combination of the appropriate density of at least 775 kg/m³, while at the same time having an end boiling point as low as 300° C., and also a freezing point of less than −47° C.

Although the hydrocarbon product stream or streams produced by the process of the present invention can be used to make a synthetic hydrocarbon fuel product according to the present invention, they can also be used in other applications which employ liquid hydrocarbons. Examples include drilling fluids, industrial solvents, printing ink solvents, metal working fluids, coatings, solvents in household product formulations, aluminium rolling, insecticide formulations, combustion fuels for portable stoves, cosmetic products, agricultural compositions, general purpose or engine degreasers, and charcoal lighter fluid.

The synthetic fuel composition of the present invention can be produced from the hydrocarbon product formed by the process of the present invention. Typically, this will be achieved by distilling and fractionating the hydrocarbon product of the process, optionally after a hydrogenation or hydrotreatment step. The synthetic hydrocarbon fuel composition has a density at 15° C. of at least 775 kg/m³ and preferably at least 780 kg/m³, and densities of 800 kg/m³ or more, or 820 kg/m³ or more are also achievable. Preferably, the density is no more than 840 kg/m³. A suitable method of measuring the density or specific gravity is described in standard ASTM D4052. The fuel composition also has a boiling point distribution having (i) a 10% recovery of 205° C. or less and (ii) an end point of 300° C. or less, for example 290° C. or less. A method of measuring the boiling point distribution is described in standard ASTM D86. The fuel composition also has a freezing point of −47° C. or less. In embodiments, the freezing point can be −60° C. or less, and in further embodiment the freezing point can be −65° C. or less. A method of measuring the freezing point is described in standard ASTM D2386.

The synthetic hydrocarbon fuel composition may have a maximum of 10% normal paraffins and a minimum of 65% monoalkyl-branched isoparaffins. Furthermore, the mixture may contain a minimum of 60% methyl-branched hydrocarbons and a maximum total of 30% hydrocarbons with 2-4 C atom branches (i.e. ethyl, propyl and butyl group branches).

Preferably, the synthetic hydrocarbon fuel composition comprises at least 90% by volume of paraffinic hydrocarbons. Preferably, the synthetic hydrocarbon fuel composition comprises less than 5% by volume unbranched paraffins.

Of the non-cyclic hydrocarbons present in the synthetic hydrocarbon fuel composition of the present invention, the majority have from 8 to 16 carbon atoms, and have predominantly from 10 to 14 carbon atoms.

In the process of the present invention, some monoaromatic compounds can be formed. For example, alkyl benzenes can be produced within the zeolite pore structure during reaction. Alkyl benzenes can be advantageous in improving seal swell properties of a resulting fuel, and also help to increase the density of hydrocarbon product streams. In addition, where hydrotreating is to be carried out, cycloalkanes can result which are useful, in that they can also improve the density of the resulting hydrocarbon product.

In one embodiment of the present invention, the synthetic hydrocarbon fuel composition comprises at least one of monocyclic aromatic and non-aromatic compounds at a combined concentration of at least 1% by volume, for example in a range of from 1 to 26.5% by volume, for example from 1 to 10% by volume, or 1 to 8% by volume. In a further embodiment their combined concentration is at least 4% by volume. A method of measuring the content of cyclic hydrocarbons is described in ASTM method D2425.

Preferably, alkylbenzenes are present at a concentration of at least 1% by volume, for example in a range of from 1 to 6% by volume, and can be present at 1.5% by volume or more.

In another embodiment, monocyclic paraffins are present in a range of from 1% to 6% by volume. In still a further embodiment, alkylbenzenes are present at a concentration of at least 1.5% by volume and monocyclic paraffins are present at a concentration of at least 4% by volume.

In one embodiment, the total concentration of cyclic hydrocarbons (i.e. aromatic and non-aromatic monocyclic and polycyclic hydrocarbons) is less than or equal to 30% by volume.

In one embodiment, the total concentration of naphthalenes is less than 3% by volume.

In one embodiment, the total concentration of aromatic compounds, as measured by ASTM D6379, is 26.5% by volume or less.

The cetane index of the synthetic hydrocarbon fuel is preferably at least 46, more preferably at least 50, and even more preferably at least 60. The cetane number of the synthetic hydrocarbon fuel composition is at least 51.

The viscosity of the synthetic hydrocarbon fuel composition at 40° C. is preferably at least 1.2 mm²/s, which can be measured by ASTM D445. Viscosities of at least 2 mm²/s at 40° C. can be achieved. The viscosity of the synthetic hydrocarbon fuel composition at −20° C. is preferably less than 8 c St.

The synthetic hydrocarbon fuel composition has a flash point of typically 55° C. or more. In the present invention, higher flash points can also be achievable, for example a flash point of 62° C. or more, and even flash points of 70° C. or more are achievable. Flash points higher than conventional jet fuels are achievable, for example, and can be associated with increased safety where fuels, or fuel-laden vehicles are stored or kept in confined spaces, such as a garage or hanger. A higher flash point is also an advantageous characteristic of diesel fuels, and hence improves compatibility of the synthetic fuel composition of the present invention for diesel engines as well as aviation jets. Flash point can be measured using method of IP170.

The present disclosure also relates to a process comprising:

a) oligomerising a mixture of alpha-olefins of the formula CH₂=CHR wherein R represents an alkyl group with 1-4 C atoms, with a propene content of no more than 30% and recycling a ratio of the recycled fraction (predominantly up to max. 170° C.) to the olefin feedstock mixture of at least 1 over a pentasil catalyst with an Si/Al ratio of >80 and a crystallite size of 0.2 to 0.8 microns in a reactor at an inlet temperature of between 180° C. and 280° C. and a pressure of at least 50 bar; and

b) hydrogenation of the oligomerised product with an initial boiling point of >160° C. with hydrogen under conventional hydrogenation conditions using conventional hydrogenation catalysts to form a saturated hydrocarbon mixture consisting of >65% monoalkyl-branched isoparaffins; and

c) taking off a distillate fraction with a density of 775 kg/m³ or more, a cetane number of at least 50, a cetane index of at least 60 and a freezing point of <−47° C.

The catalyst can contain 1-10% of an oxide of a lanthanum group element.

The WHSV can be in the range of from 0.4 to 1.

The recyclate/fresh feedstock ratio can be >1.

The process can be in accordance with the process of the present invention, and the resulting product can be used to prepare the synthetic hydrocarbon fuel composition of the present invention.

### EXAMPLES

The invention will now be illustrated by the following non-limiting examples, with reference to the drawings in which:

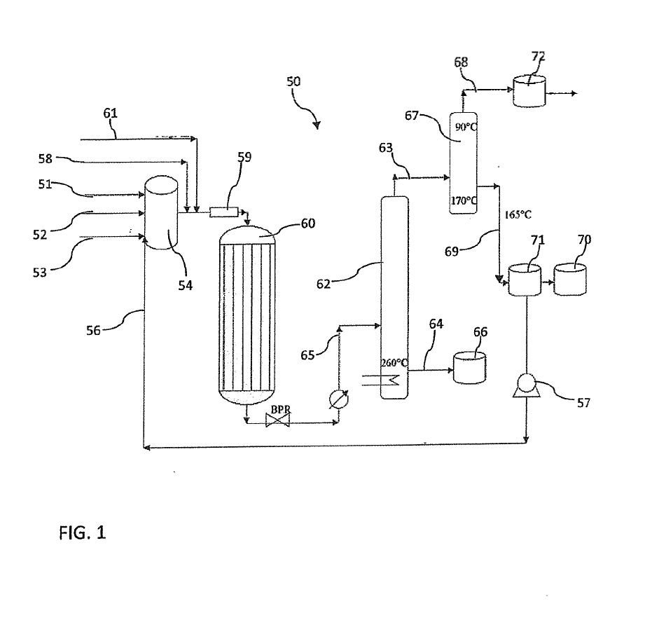

FIG. 1 is a schematic representation of a manufacturing plant in which olefins are oligomerised according to the present invention to produce a hydrocarbon composition;

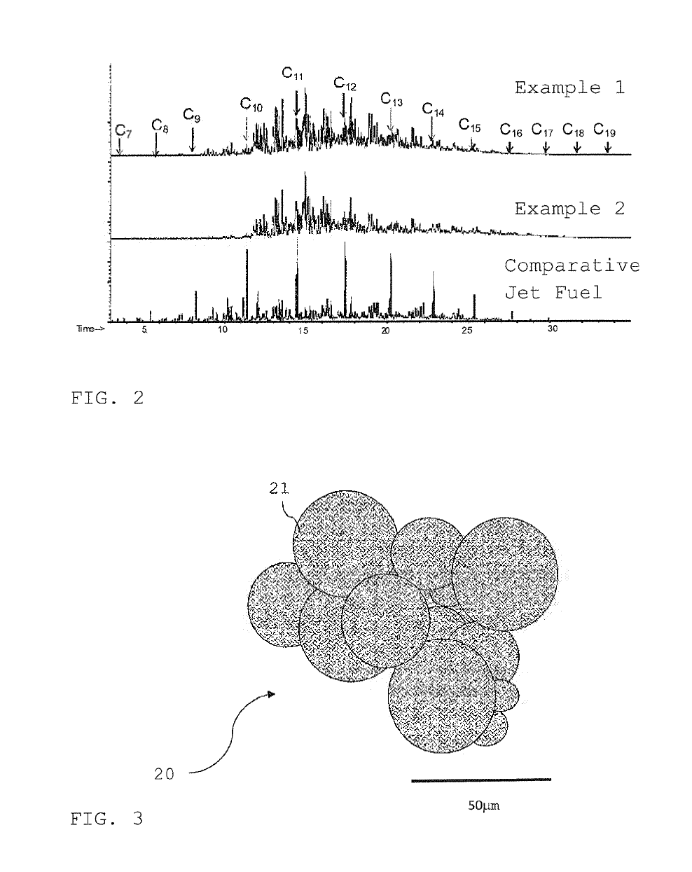

FIG. 2 compares three GC traces of a conventional crude oil-derived jet fuel, a synthetic hydrocarbon fuel according to the present invention, and a synthetic prior art hydrocarbon fuel; and

FIG. 3 illustrates agglomerated zeolite crystals.

In the Tables, the following compositions are compared. Examples 1 and 2 are synthetic hydrocarbon fuels according to the present invention, and obtained as a fraction in the method according to the present invention, as outlined in further detail below. The difference between Examples 1 and 2 is the boiling point range of the fraction, example 2 having a slightly higher end point of the boiling range.

Comparative Example 1 is a synthetic jet fuel, obtained from hydrocarbons obtained from a Fischer-Tropsch process, and not via olefin oligomerisation.

Conventional Jet Fuels A, B, C and D are four different fuels derived from crude oil refining.

Table 1 lists comparative data of the fuel compositions. Of note is that the synthetic hydrocarbon fuels of the present invention have both a freezing point of below −47° C. and also a specific gravity at 15° C. of significantly greater than 0.775 (equivalent to a density of 775 kg/m³). The comparative synthetic fuel does not meet both of these requirements, the specific gravity being lower than 0.775.

Of further note is the flash point of Examples 1 and 2. The values are substantially higher than the comparison fuels, even the fuels derived from conventional crude oil sources.

Table 2 shows comparison of the synthetic fuels of Examples 1 and 2 and Comparative Example 1 with data averaged over several crude oil-derived jet fuels. Also shown is the specification for jet fuel JP-8 for comparison. The same trends outlined above are observed. Of note is that the synthetic fuel of the present invention contains aromatic compounds, whereas the comparative synthetic fuel does not. The aromatics content helps to increase the density of the synthetic fuel and improve seal swell capacity.

Another observation is that the viscosity @40° C. of Example 1, of 1.436 mm²/s, is within the specification required for a diesel fuel, again demonstrating its usefulness as both a diesel and aviation jet fuel.

Table 3 shows some compositional data of various fuels, including Examples 1 and 2, Comparative Example 1, and Conventional Jet Fuel A.

TABLE 1
Comparative data of fuel compositions

| | | | Comparative | Conventional Jet Fuel | | | |
|---|---|---|---|---|---|---|---|
| Test | Example 1 | Example 2 | Example 1 | A | B | C | D |
| Total Acid Number, | 0.002 | 0.004 | 0.004 | 0.003 | | | |
| mg KOH/g | | | | | | | |
| Aromatics, vol % | 5.2 | 3.3 | 0.0 | 18.8 | 17.2 | 20.3 | 16.9 |
| Olefins, vol % | 1.2 | 1.1 | 0.0 | 0.8 | 0.5 | 0.6 | 1.1 |
| Mercaptan Sulfur, % | 0.000 | NA | 0.000 | 0.000 | | | |
| mass | | | | | | | |
| Total Sulfur, % mass | 0.00 | NA | 0.00 | 0.04 | | | |
| Distillation: | | | | | | | |
| IBP, ° C. | 192 | 194 | 144 | 159 | 152 | 160 | 162 |
| 10% recovered, ° C. | 200 | 202 | 167 | 182 | 173 | 177 | 183 |
| 20% recovered, ° C. | 203 | 206 | 177 | 189 | 179 | 183 | 190 |
| 50% recovered, ° C. | 212 | 215 | 206 | 208 | 198 | 200 | 207 |
| 90% recovered, ° C. | 240 | 257 | 256 | 244 | 239 | 237 | 237 |
| EP, ° C. | 264 | 278 | 275 | 265 | 260 | 255 | 265 |
| Residue, % vol | 1.2 | 1.3 | 1.5 | 1.3 | 1.1 | 1.2 | 1.2 |
| Loss, % vol | 0.6 | 0.9 | 0.9 | 0.8 | 0.2 | 0.7 | 0.7 |
| Flash point, ° C. | 71 | 74 | 45 | 51 | 48 | 52 | 52 |
| Cetane Index | 64.9 | 61.8 | 66.0 | 46.0 | | | |
| (calculated) | | | | | | | |
| Freeze Point, ° C. | <−78 | −78 | −51 | −50 | −50 | −49 | −58 |
| Viscosity @ −20° C., cSt | 6.5 | 7.2 | 4.9 | 4.9 | | | |
| Viscosity @ −40° C., cSt | 15.1 | 18.4 | 9.5 | 9.9 | | | |
| Heat of Combustion | 18900 | 18800 | 18900 | 18600 | 18500 | 18500 | 18500 |
| (measured), BTU/lb | | | | | | | |
| Hydrogen Content, % | 14.8 | 14.8 | 15.4 | 13.8 | | | |
| mass | | | | | | | |
| Smoke Point, mm | 40 | NA | 42 | 22 | | | |
| Copper Strip | 1a | 1a | 1a | 1a | | | |
| Corrosion | | | | | | | |
| Thermal Stability @ | | | | | | | |
| 260° C.: | | | | | | | |
| Tube Deposit Rating | 1 | NA | 1 | 1 | | | |
| Change in Pressure, | 0 | NA | 0 | 2 | | | |
| mm Hg | | | | | | | |
| Existent Gum, | <1 | NA | 0.6 | 0.4 | | | |
| mg/100 mL | | | | | | | |
| API Gravity @ 60° F. | 49.8 | 49.7 | 55.6* | 44.4 | 45.6 | 45.7 | 42.2 |
| Specific Gravity @ | 0.780 | 0.781 | 0.756* | 0.804 | 0.799 | 0.798 | 0.815 |
| 15° C. (D4052) | | | | | | | |

NA = Not analysed
*Value outside specification limit

TABLE 2
Further comparative data of fuel compositions

| | | | Comparative | Conventional Jet Fuel | |
|---|---|---|---|---|---|
| Specification Test | Example 1 | Example 2 | Example 1 | Average | Spec |
| Paraffins (normal + iso), vol % | 90 | 91 | >99 | ~60 (+~20% | |
| | | | | cycloparaffins) | |
| Aromatics, vol % (D1319) | 5.2 | 3.3 | 0.0 | 17.9 | ≦25 |
| Specific gravity (D4052) | 0.780 | 0.781 | 0.753* | 0.803 | 0.775-0.840 |
| Flash point, C. (D93) | 71 | 74 | 45 | 49 | ≧38 |
| Freeze point, C. (D5972) | <−78 | −78 | −51 | −51.5 | ≦−47 |
| Viscosity @40° C. MM²/s ASTM | 1.436 | NA | NA | NA | NA |
| D445 | | | | | |
| Hydrogen content (D3343), mass | 14.8 | 14.8 | 15.4 | 13.8 | ≧13.4 |
| % | | | | | |
| Heat of combustion, MJ/kg | 43.9 | 43.7 | 44.1 | 43.2 | ≧42.8 |
| (D4809) | | | | | |
| Smoke point, mm | 40 | NA | 42 | 23 | ≧19 |

NA = Not analysed
*Value outside specification limit

TABLE 3
Product Characteristics of Fuel Compositions

| | | | | Conventional |
|---|---|---|---|---|
| | | | | Jet |
| | | | Comparative | Fuel |
| Specification Test | Example 1 | Example 2 | Example 1 | A |
| D2425 (vol. %) | | | | |
| Paraffins | 90 | 91 | >99 | 60 |
| (normal + iso) | | | | |
| Cycloparaffins | 4.1 | 4.2 | <1 | 15 |
| Dicycloparaffins | 1.9 | 3.2 | <1 | 5.9 |
| Tricycloparaffins | <1 | <1 | <1 | <1 |
| Alkylbenzenes | 3.3 | 1.6 | <0.5 | 12 |
| Indans and Tetralins | <0.5 | <0.5 | <0.5 | 6.3 |
| Indenes and | <0.5 | <0.5 | <0.5 | <0.5 |
| CₙH₂ₙ₋₁₀ | | | | |
| Naphthalene | <0.5 | <0.5 | <0.5 | <0.5 |
| Naphthalenes | <0.5 | <0.5 | <0.5 | 1.2 |
| Acenaphthenes | <0.5 | <0.5 | <0.5 | <0.5 |
| Acenaphthylenes | <0.5 | <0.5 | <0.5 | <0.5 |
| Tricyclic Aromatics | <0.5 | <0.5 | <0.5 | <0.5 |
| Total | 100 | 100 | 100 | 100 |

The synthetic fuels of the present invention have a higher total paraffin content compared to the crude oil-derived conventional jet fuel, but less than the content of the comparative synthetic fuel. The fuels of Examples 1 and 2 have a higher cyclic hydrocarbon content compared to the comparative synthetic fuel, which contribute to increased density.

The jet fuels described of Examples 1 and 2 were produced in a pilot plant, 50, schematically represented in FIG. 1.

Liquefied propene, 51, and butene, 52, and a mixture of pentenes and hexenes, 53, were introduced to the mixing chamber, 54, via cooled metering pumps (not shown). Hydrogen, 58, was introduced downstream of the propene, 51, and 1-butene, 52, feed.

The mixture of olefins and hydrogen was combined, pressurised and preheated to 220° C. in a preheater, 59, and fed into the reactor, 60.

A recycle line, 56, recycled product back to the reactor, 60, via the mixing chamber, 54, by means of a cooled recycle pump, 57. The recycle pump stroke was adjusted to ensure a recycle to fresh feed ratio of 1:1 to 2.5 by weight.

The tubular reactor, 60, was jacketed in an electrically heated salt bath, although other heating equipment such as a 3-zone heater could alternatively be used. The maximum operating temperature for the reaction was 300° C., although temperatures as high as 480° C. are possible where catalyst regeneration or pre-treatment is to take place. The temperature at the beginning of reaction is 230° C., and is increased gradually to 280° C. in order to compensate for gradual loss of catalyst activity. The operating pressure in the reactor was maintained at between 50 to 60 bar (5.0 to 6.0 MPa). Hydrogen, 58, was fed at a concentration of 5% mol compared to the fresh feed. Hydrogen helps to avoid catalyst coking and improve catalyst lifetime. Its concentration was controlled, however, to avoid excessive hydrogenation of olefins, which would tend to inhibit the reaction rate and the extent of oligomerisation. The fresh feed comprised propylene, 51, 1-butene, 52, and a mixture of pentene and hexane, 53. The weight hourly space velocity of the feedstock was 0.5 h⁻¹.

The purge line, 61, was used for feeding nitrogen (inert) or nitrogen plus oxygen for regenerating the catalyst.

Before reaction, the catalytic reactor 60 is preheated at 230° C. before allowing the feed streams into the reactor. After obtaining a stable temperature of 230° C. and a stable pressure of 60 bar (6.0 MPa) within the reactor, the recyclate, 56, and the pentene and hexene mixture, 53, were introduced into the reactor, 60. Once the catalyst bed temperature and other process variables were stable, the propene, 51, and butane, 52, were fed into the system.

The reactor outlet stream, 65, was cooled to a temperature of 180° C.-200° C. and fed to a distillate splitter at a pressure of 1 bar (100 kPa), 62, to produce a light fraction, 63, and a middle distillate fraction, 64. The middle distillate fraction had an initial boiling point of 180° C. at atmospheric pressure. The middle distillate stream was collected in storage vessel 66.

The light fraction, 63, was fed to a further distillation column, 67, where a vapour fraction, 68, was separated from a liquid fraction, 69. The vapour fraction, 68, contained vaporous hydrocarbons plus unreacted hydrogen. The liquid fraction, 69, contained liquid hydrocarbons in the C₅ to C₉ range. This light liquid hydrocarbon product, 69, with a boiling range of 160 to 170° C. at 100 kPa, was fed to a buffer vessel, 71, equipped with level control. A portion of the liquid product was collected in a second storage vessel, 70, at ambient temperature and pressure. This product is suitable for use as, or for blending with, gasoline.

A portion of the liquid fraction, 69, was used as a recyclate, 56, and returned to the reactor, 60.

The vapour fraction, 68, from distillation column 67, was withdrawn at a temperature of 90-100° C. at 100 kPa, and routed to a separator, 72, with pressure control. An online gas chromatograph (not shown) was used to monitor the propene content of this stream to calculate propene conversion. The propene conversion was maintained at 98-99%.

Additional product properties were determined by gas chromatography coupled to a mass spectrometer and the branching of the products was determined by NMR.

The compositions of Examples 1 and 2 were collected from the middle distillate fraction, 64, of the process referred to above.

FIG. 2 shows GC traces of fuel compositions referred to in the tables above, showing hydrocarbon distributions based on numbers of carbon atoms. Examples 1 (top trace) and 2 (middle trace) have a high proportion of hydrocarbons in the C10 to C14 range. In addition, there is a more varied spread of hydrocarbon types, indicating the presence of a range of branched and unbranched hydrocarbons. The hydrocarbons of the comparative fuel (bottom trace), from a conventional crude oil-derived jet fuel, has a broader range of hydrocarbon sizes (C9 to C16), and has major peaks corresponding to unbranched hydrocarbons, highlighting the lower extent of hydrocarbon branching.

FIG. 3 illustrates agglomerated zeolite crystals that can be formed by a method outlined below. The agglomerated particle, 20, comprises a number of individual zeolite crystals (or crystallites), 21, bonded together. Voids, or meso and macropores, are formed between the crystallites, 21, and within the agglomerated particle, 20, that allow transfer of reactants and products to the zeolite crystals, 21, in the interior of the particles 20.

From an aqueous reaction batch containing a source of silicon, a source of aluminium, a source of alkali and a template, an alkaline aluminosilicate gel is produced as described in U.S. Pat. No. 3,702,886. At elevated temperature and pressure, a crystalline aluminosilicate is formed. A surface-tension modifier is also added to aid the formation of particle aggregates. The reaction is stopped when the primary crystallites obtained have a mean diameter between 0.1 microns to 0.9 microns, preferably in the range of 0.2 to 0.6 microns. The aggregated primary crystallites are recovered from solution, dried and calcined, first in an inert atmosphere, and subsequently in an oxidizing atmosphere at a temperature of 500° to 600° C. The intermediate product is then ion exchanged by contacting with an ammonium salt solution, whereby the alkali metal ions are exchanged for ammonium ions. The agglomerated crystalline and ion-exchanged product is recovered by filtration, dried and subjected to further calcination to remove the ammonia to obtain a protonated zeolite catalyst in powder form. The powder can be ground in a laboratory mill to the required agglomerated particle size, for example a particle size of <500 microns, before use.

1. A process for producing hydrocarbons, comprising contacting one or more olefins with an oligomerisation catalyst in a reaction zone under conditions sufficient to cause oligomerisation of olefins, and removing from the reaction zone an outlet stream comprising products of olefin oligomerisation, wherein a fraction is obtained from the outlet stream which, optionally after hydrotreatment, has the following properties:
a) a boiling point distribution having (i) a 10% recovery of 205° C. or less and (ii) an end point of 300° C. or less;
b) a freezing point of −47° C. or less; and
c) a density at 15° C. of at least 775.0 kg/m³.

2. A process according to claim 1, in which the oligomerisation catalyst is a crystalline zeolite Brønsted acid catalyst.

3. A process according to claim 2, in which the zeolite crystals comprise an inorganic oxide framework with an internal pore structure, and an external surface, in which the ratio of the number of Brønsted acid sites on the external surface compared to the internal pore structure is in the range of from 0.1 to 20% and/or the ratio of the surface area of the internal pore structure compared to the external crystal surface area is in the range of from 5 to 1000.

4. A process for producing hydrocarbons, comprising contacting one or more olefins with an oligomerisation catalyst in a reaction zone under conditions sufficient to cause oligomerisation of olefins, and removing from the reaction zone an outlet stream comprising products of olefin oligomerisation, wherein the oligomerisation catalyst comprises a crystalline zeolite Brønsted acid catalyst, whose crystals comprise an inorganic oxide framework with an internal pore structure, and an external surface, in which the ratio of the number of Brønsted acid sites on the external surface compared to the internal pore structure is in the range of from 0.1 to 20% and/or the ratio of the surface area of the internal pore structure compared to the external crystal surface area is in the range of from 5 to 1000.

5. A process as claimed in claim 4, in which a fraction is obtained from the outlet stream which, optionally after hydrotreatment, has the following properties:
a) a boiling point distribution having (i) a 10% recovery of 205° C. or less and (ii) an end point of 300° C. or less;
b) a freezing point of −47° C. or less; and
c) a density at 15° C. of at least 775.0 kg/m³.

6. A process as claimed in claim 2, in which the zeolite is an aluminosilicate.

7. A process as claimed in claim 6, in which the Si/Al molar ratio of the aluminosilicate zeolite is in the range of from 45 to 300; and/or
the zeolite adopts the MFI structure, and is preferably ZSM-5; and/or
the average primary crystal size of the zeolite is in the range of from 0.1 to 0.9 μm.

8-9. (canceled)

10. A process as claimed in claim 1, in which the olefins are derived from a Fischer-Tropsch process or from an alcohol dehydration process; and/or
the reaction zone is maintained at a temperature in the range of from 180 to 350° C.; and/or
a fraction boiling at a temperature of 180° C. or less is separated from the outlet stream, at least a portion of which is recycled to the reaction zone; and/or
any outlet stream or fraction obtained from the outlet stream undergoes hydrotreatment.

11-13. (canceled)

14. A hydrocarbon composition obtainable by the process according to claim 1.

15. A synthetic hydrocarbon fuel composition having the following properties:
a) a boiling point distribution having (i) a 10% recovery of 205° C. or less and (ii) an end point of 300° C. or less;
b) a freezing point of −47° C. or less;
c) a density at 15° C. of at least 775.0 kg/m³; and
d) a total concentration of cyclic hydrocarbons of less than or equal to 30% by volume.

16. A synthetic hydrocarbon fuel composition as claimed in claim 15, having a flash point of at least 55° C.

17. (canceled)

18. A synthetic hydrocarbon fuel composition as claimed in claim 15, in which the density is at most 840.0 kg/m³; and/or
the freezing point is −60° C. or less; and/or
the density at 15° C. is at least 780 kg/m³; and/or
the boiling point distribution end point is 290° C. or less.

19-21. (canceled)

22. A synthetic hydrocarbon fuel composition as claimed in claim 15, having:
a combined concentration of monoaromatic compounds and monocyclic paraffins of at least 1% by volume; and/or
a content of n-paraffins of no more than 10% by volume; and/or
a paraffin content of at least 90% by volume; and/or
a content of monoalky-branched iso-paraffins of at least 65% by volume and/or a content of methyl-branched iso-paraffins of at least 60% by volume; and/or
a content of alkylbenzenes in the range of from 1 to 6% by volume; and/or
a viscosity at 40° C. of at least 1.2 mm²/s; and/or
a viscosity at −20° C. of 8 cSt or less; and/or
a cetane number of at least 51 and/or a cetane index of at least 46.

23-30. (canceled)

31. A hydrocarbon fuel blend comprising the synthetic hydrocarbon fuel composition as claimed in claim 15.

32. A jet fuel or diesel fuel comprising the synthetic hydrocarbon fuel composition as claimed in claim 15.

33. (canceled)

34. A process as claimed in claim 4, in which the zeolite is an aluminosilicate.

35. A process as claimed in claim 35, in which the Si/Al molar ratio of the aluminosilicate zeolite is in the range of from 45 to 300; and/or
the zeolite adopts the MFI structure, and is preferably ZSM-5; and/or;
the average primary crystal size of the zeolite is in the range of from 0.1 to 0.9 μm.

36. A process as claimed in claim 4, in which the olefins are derived from a Fischer-Tropsch process or from an alcohol dehydration process; and/or
the reaction zone is maintained at a temperature in the range of from 180 to 350° C.; and/or
a fraction boiling at a temperature of 180° C. or less is separated from the outlet stream, at least a portion of which is recycled to the reaction zone; and/or
any outlet stream or fraction obtained from the outlet stream undergoes hydrotreatment.

37. A hydrocarbon composition obtainable by the process according to claim 4.
